(19) United States
(12) Patent Application Publication
(10) Pub. No.: US 2005/0238699 A1
(43) Pub. Date: Oct. 27, 2005

(54) Fibrous structures comprising a surface treating composition and lotion composition

(76) Inventor: Joerg Kleinwaechter, Hofheim Am Tanus (DE)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/105,976

(22) Filed: Apr. 14, 2005

Related U.S. Application Data
(60) Provisional application No. 60/657,230, filed on Feb. 28, 2005.
(60) Provisional application No. 60/565,104, filed on Apr. 23, 2004.
(60) Provisional application No. 60/565,105, filed on Apr. 23, 2004.

(51) Int. Cl. A61K 9/70

(52) U.S. Cl. 424/443

(57) ABSTRACT

Fibros structures comprising a surface treating composition and a lotion composition, products made therefrom and processes for making same are provided. More particularly, fibrous structures comprising a user contacting surface comprising a first region comprising a surface treating composition and a second region comprising a lotion composition are provided.

### CROSS REFERENCE TO RELATED APPLICATIONS

This application claims the benefit of U.S. Provisional Application Ser. No. 60/657,230 filed on Feb. 28, 2005 and U.S. Provisional Application Ser. No. 60/565,104 filed on Apr. 23, 2004 and U.S. Provisional Application Ser. No. 60/565,105 filed on Apr. 23, 2004.

### FIELD OF THE INVENTION

The present invention relates to fibrous structures comprising a surface treating composition and a lotion composition, single- or multi-ply sanitary tissue products made therefrom and processes for making same. More particularly, the present invention relates to fibrous structures comprising a user contacting surface comprising a first region and a second region, wherein the first region comprises a first composition and the second region comprises a second composition different from the first composition. Even more particularly, the present invention relates to fibrous structures comprising a user contacting surface comprising a first region comprising a surface treating composition and a second region comprising a lotion composition.

### BACKGROUND OF THE INVENTION

Fibrous structures comprising a surface treating composition such as a softening composition and a lotion composition are known. However, such conventional fibrous structures have utilized full coverage of a surface of the fibrous structure with the surface treating compositions and/or lotion compositions in an attempt to maximize transfer of the lotion composition.

Formulators have added an anti-migration material, such as a quaternary ammonium compound, into a fiber furnish such that the fibers are coated with the quaternary ammonium compound. The fibrous structure formed by the fiber furnish tends to mitigate the migration of a subsequently applied lotion composition into the fibrous structure. The function of the quaternary ammonium compound and the level at which it is used are not provide softening of the fibrous structure surface.

It is also known in the art to add quaternary ammonium compounds and/or silicones and/or other types of agents into the fiber furnish for the purpose of debonding the fibers.

None of the known fibrous structures teach or suggest treating a surface of the fibrous structure with a surface treating composition and a lotion composition such that a user contacting surface comprising a first region comprising the surface treating composition and a second region comprising a lotion composition is produced on the surface of the fibrous structure.

Accordingly, there is a need for a fibrous structure that comprises a surface treating composition such as a softening composition and a lotion composition such that a user contacting surface comprising a first region comprising the surface treating composition and a second region comprising the lotion composition is produced on a surface of the fibrous structure, a single- or multi-ply sanitary tissue product made therefrom and processes for making same.

### SUMMARY OF THE INVENTION

The present invention fulfills the needs described above by providing a fibrous structure and/or sanitary tissue product comprising a surface treating composition and a lotion composition such that a user contacting surface comprising a first region comprising the surface treating composition and a second region comprising the lotion composition is produced on a surface of the fibrous structure and/or sanitary tissue product.

In one example of the present invention, a fibrous structure and/or single- or multi-ply sanitary tissue product comprising a user contacting surface wherein the user contacting surface comprises a first region comprising a surface treating composition and second region comprising a lotion composition is provided.

In another example of the present invention, a fibrous structure and/or single- or multi-ply sanitary tissue product comprising a surface treating composition and a lotion composition, wherein the surface treating composition is present on a surface of the fibrous structure and/or single- or multi-ply sanitary tissue product at a greater level by weight than within the fibrous structure and/or single- or multi-ply sanitary tissue product and the lotion composition is present within the fibrous structure and/or single- or multi-ply sanitary tissue product at a greater level by weight than on the surface of the fibrous structure and/or single- or multi-ply sanitary tissue product, is provided.

Relative concentration of the surface treating composition and/or lotion composition on the surface can be determined using the Relative Concentration on Surface Test Method described herein.

In yet another example of the present invention, a fibrous structure and/or single- or multi-ply sanitary tissue product comprising:

a. a surface treating composition comprising a surface treating agent selected from the group consisting of: polymers, hydrocarbons, waxes, oils, silicones, quaternary ammonium compounds, fluorocarbons, substituted C₁₀-C₂₂ alkanes, substituted C₁₀-C₂₂ alkenes, polyols, sugar derivatives and mixtures thereof; and

b. a lotion composition comprising a compound selected from the group consisting of: oils, alcohol ethoxylates, fatty acid esters, hydrocarbons and mixtures thereof;

wherein the surface treating composition is present on a surface of the fibrous structure and/or single- or multi-ply sanitary tissue product and the lotion composition is present on less than the entire surface of the surface treating composition, is provided.

In even another example of the present invention, a single- or multi-ply sanitary tissue product comprising a fibrous structure according to the present invention is provided.

In even yet another example of the present invention, a process for treating a fibrous structure comprising the step of applying a lotion composition to a surface treating composition associated with a surface of a fibrous structure and/or single- or multi-ply sanitary tissue product, is provided.

In still yet another example of the present invention, a process for treating a fibrous structure and/or single- or multi-ply sanitary tissue product comprising the steps of:

a. applying a surface treating composition to a surface of a fibrous structure and/or single- or multi-ply sanitary tissue product; and

b. applying a lotion composition to the surface treating composition, is provided.

In even still yet another example of the present invention, a process for treating a fibrous structure and/or single- or multi-ply sanitary tissue product comprising the steps of:

a. applying a surface treating composition to a surface of a fibrous structure and/or single- or multi-ply sanitary tissue product; and

b. applying a lotion composition to the surface of the fibrous structure and/or single- or multi-ply sanitary tissue product, is provided.

Accordingly, the present invention provides fibrous structures and/or single- or multi-ply sanitary tissue products comprising a surface treating composition and a lotion composition, products made therefrom and processes for making same.

### BRIEF DESCRIPTION OF THE DRAWINGS

FIG. 1 is a schematic representation of a fibrous structure in accordance with the present invention;

FIG. 2 is a cross-sectional view of FIG. 1 taken along line 2-2;

FIG. 3 is a cross-sectional view of another example of a fibrous structure in accordance with the present invention;

FIG. 4 is a cross-sectional view of another example of a fibrous structure in accordance with the present invention;

FIG. 5 is a cross-sectional view of another example of a fibrous structure in accordance with the present invention;

FIG. 6 is a schematic representation of another example of a fibrous structure in accordance with the present invention.

### DETAILED DESCRIPTION OF THE INVENTION

Definitions

“Fiber” as used herein means an elongate particulate having an apparent length greatly exceeding its apparent diameter, i.e. a length to diameter ratio of at least about 10. Fibers having a non-circular cross-section are common; the “diameter” in this case may be considered to be the diameter of a circle having cross-sectional area equal to the cross-sectional area of the fiber. More specifically, as used herein, “fiber” refers to papermaking fibers. The present invention contemplates the use of a variety of papermaking fibers, such as, for example, natural fibers or synthetic fibers, or any other suitable fibers, and any combination thereof.

Natural papermaking fibers useful in the present invention include animal fibers, mineral fibers, plant fibers and mixtures thereof. Animal fibers may, for example, be selected from the group consisting of: wool, silk and mixtures thereof. Plant fibers may, for example, be derived from a plant selected from the group consisting of: wood, cotton, cotton linters, flax, sisal, abaca, hemp, hesperaloe, jute, bamboo, bagasse, kudzu, corn, sorghum, gourd, agave, loofah and mixtures thereof.

Wood fibers; often referred to as wood pulps include chemical pulps, such as kraft (sulfate) and sulfite pulps, as well as mechanical and semi-chemical pulps including, for example, groundwood, thermomechanical pulp, chemi-mechanical pulp (CMP), chemi-thermomechanical pulp (CTMP), neutral semi-chemical sulfite pulp (NSCS). Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified and/or layered fibrous structure. U.S. Pat. No. 4,300,981 and U.S. Pat. No. 3,994,771 are incorporated herein by reference for the purpose of disclosing layering of hardwood and softwood fibers. Also applicable to the present invention are fibers derived from recycled paper, which may contain any or all of the above categories as well as other non-fibrous materials such as fillers and adhesives used to facilitate the original papermaking.

The wood pulp fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. Softwood fibers derived from the kraft process and originating from more-northern climates may be preferred. These are often referred to as northern softwood kraft (NSK) pulps.

Synthetic fibers may be selected from the group consisting of: wet spun fibers, dry spun fibers, melt spun (including melt blown) fibers, synthetic pulp fibers and mixtures thereof. Synthetic fibers may, for example, be comprised of cellulose (often referred to as “rayon”); cellulose derivatives such as esters, ether, or nitrous derivatives; polyolefins (including polyethylene and polypropylene); polyesters (including polyethylene terephthalate); polyamides (often referred to as “nylon”); acrylics; non-cellulosic polymeric carbohydrates (such as starch, chitin and chitin derivatives such as chitosan); and mixtures thereof.

“Fibrous structure” as used herein means a structure that comprises one or more fibers. Nonlimiting examples of processes for making fibrous structures include known wet-laid papermaking processes and air-laid papermaking processes. Such processes typically include steps of preparing a fiber composition, oftentimes referred to as a fiber slurry in wet-laid processes, either wet or dry, and then depositing a plurality of fibers onto a forming wire or belt such that an embryonic fibrous structure is formed, drying and/or bonding the fibers together such that a fibrous structure is formed, and/or further processing the fibrous structure such that a finished fibrous structure is formed. For example, in typical papermaking processes, the finished fibrous structure is the fibrous structure that is wound on the reel at the end of papermaking, but before converting thereof into a sanitary tissue product.

“Sanitary tissue product” comprises one or more fibrous structures, converted or not, that is useful as a wiping implement for post-urinary and post-bowel movement cleaning (toilet tissue), for otorhinolaryngological discharges (facial tissue and/or disposable handkerchiefs), and multi-functional absorbent and cleaning uses (absorbent towels and/or wipes). In one example, a lotion composition-containing multi-ply disposable handkerchief having a caliper of from about 0.1 mm to about 0.4 mm in accordance with the present invention is provided.

“Ply” or “Plies” as used herein means an individual finished fibrous structure optionally to be disposed in a substantially contiguous, face-to-face relationship with other plies, forming a multiple ply finished fibrous structure product and/or sanitary tissue product. It is also contemplated that a single fibrous structure can effectively form two “plies” or multiple “plies”, for example, by being folded on itself.

“Surface of a fibrous structure” as used herein means that portion of the fibrous structure that is exposed to the external environment. In other words, the surface of a fibrous structure is that portion of the fibrous structure that is not completely surrounded by other portions of the fibrous structure.

“User Contacting Surface” as used herein means that portion of the fibrous structure and/or surface treating composition and/or lotion composition present directly and/or indirectly on the surface of the fibrous structure that is exposed to the external environment. In other words, it is that surface formed by the fibrous structure including any surface treating composition and/or lotion composition present directly and/or indirectly on the surface of the fibrous structure that contacts an opposing surface when used by a user. For example, it is that surface formed by the fibrous structure including any surface treating composition and/or lotion composition present directly and/or indirectly on the surface of the fibrous structure that contacts a user's skin when a user wipes his/her skin with the fibrous structure of the present invention.

In one example, the user contacting surface, especially for a textured and/or structured fibrous structure, such as a through-air-dried fibrous structure and/or an embossed fibrous structure, may comprise raised areas and recessed areas of the fibrous structure. In the case of a through-air-dried, pattern densified fibrous structure the raised areas may be knuckles and the recessed areas may be pillows and vice versa. Accordingly, the knuckles may, directly and/or indirectly, comprise the lotion composition and the pillows may comprise the surface treating composition and vice versa so that when a user contacts the user's skin with the fibrous structure, the lotion composition and surface treating composition both contact the user's skin. A similar case is true for embossed fibrous structures with the embossed areas may, directly and/or indirectly, comprise the lotion composition and the non-embossed areas may comprise the surface treating composition and vice versa.

In one example, the user contacting surface has to comprise regions of sufficient size such that two or more different regions (comprising different compositions) are exposed to an opposing surface during use. In other words, a surface of a fibrous structure that is substantially covered (on a microscopic scale) by a lotion composition but completely covered on a macro scale by such lotion composition such that a user's skin is only contacted by the lotion composition does not contain two different regions in its user contacting surface. In one example a user contacting surface may comprise an external layer of a multi-layer fibrous structure wherein the external layer may comprise a surface treating composition and/or a lotion composition.

The user contacting surface may be present on the fibrous structure and/or sanitary tissue product before use by the user and/or the user contacting surface may be created/formed prior to and/or during use of the fibrous structure and/or sanitary tissue product by the user, such as upon the user applying pressure to the fibrous structure and/or sanitary tissue product as the user contacts the user's skin with the fibrous structure and/or sanitary tissue product.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

Fibrous Structure

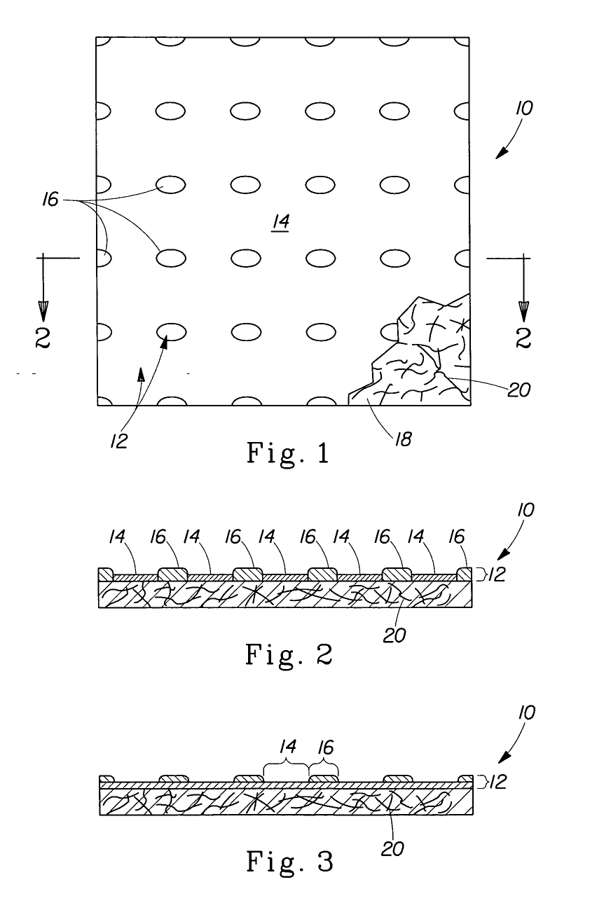

FIG. 1 is a schematic representation of a fibrous structure in accordance with the present invention. As shown in FIG. 1, a fibrous structure 10 comprising a user contacting surface 12 comprising a first region 14 and a second region 16. The user contacting surface 12 is associated with a surface of the fibrous structure 18. As shown, the surface of the fibrous structure 18 may comprise one or more fibers 20.

The first region 14 and/or second region 16 may be present on (associated with) the surface of the fibrous structure 18. When the first region 14 and/or the second region 16 are present on the surface of the fibrous structure 18, one or both may be present on the surface of the fibrous structure 18 in the form of a continuous or substantially continuous network and/or in a plurality of discrete areas (also sometimes known as “islands”).

When present on the surface of the fibrous structure 18, the first region 14 and/or the second region 16 may be in contact with and/or cover the entire or substantially the entire surface area of the surface of the fibrous structure 18. In one example, the first region 18 is in contact with and/or covers the entire or substantially the entire surface area of the surface of the fibrous structure 18.

When present on the surface of the fibrous structure 18, the first region 14 and/or the second region 16 may be in contact with and/or cover less than the entire or substantially the entire surface area of the surface of the fibrous structure 18. In one example, the second region 16 is in contact with and/or covers less than the entire or substantially the entire surface area of the surface of the fibrous structure 18. When either region covers less than substantially the entire surface area of the surface of the fibrous structure 18, that region may be in the form of a plurality of discrete areas.

As shown in FIG. 2, the first region 14 is in contact with and/or covers substantially the entire surface area of the surface of the fibrous structure 18 and the second region 16 is in contact with and/or covers less than substantially the entire surface area of the surface of the fibrous structure 18. The first region 14 may be in the form of a continuous or substantially continuous network and the second region 16 may be in the form of a plurality of discrete areas dispersed throughout the continuous or substantially continuous network of the first region 14.

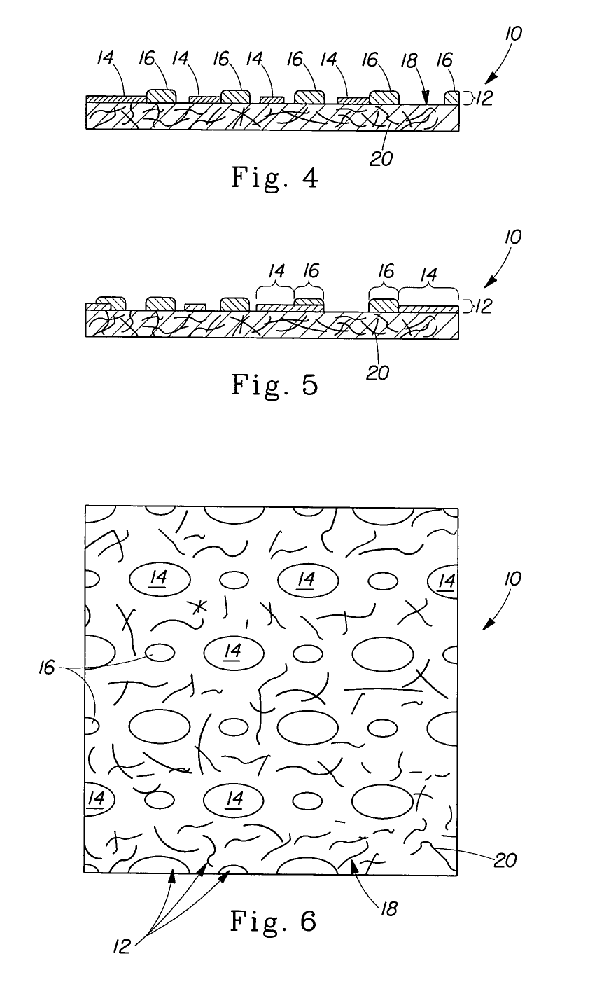

Either region may be in contact with the other region. As shown in FIG. 3, the second region 16 is in contact with the first region 14 such that the first region 14 is positioned between the second region 16 and the surface of the fibrous structure 18. The second region 16 may be present on less than the entire surface area of the first region 14. The second region 16 may be present on the first region 14 in the form of one or more discrete areas. As shown in FIGS. 1, 4 and 5, portions of the second region 16 are in contact with the first region 14 such that both the second region 16 and the first region 14 are in contact directly with the surface of the fibrous structure 18. Also as shown in FIGS. 4 and 5, a portion of the second region 16 is not in contact with the first region 14.

FIGS. 4 and 5 also show that less than substantially the entire surface area of the surface of the fibrous structure 18 is in contacted by or covered by the first region 14 and the second region 16. In these examples, the user contacting surface 12 comprises a third region, namely, the surface of the fibrous structure 18 as well as the first region 14 and the second region 16.

FIG. 6 is a schematic representation of another example of a fibrous structure in accordance with the present invention. The fibrous structure 10 comprises a user contacting surface 12 that comprises a first region 14, a second region 16 and a third region, in this case, the surface of the fibrous structure 18 which comprises one or more fibers 20.

The first region 14 comprises a surface treating composition.

The second region 16 comprises a lotion composition.

In one example, the surface treating composition and/or lotion composition may be present on the surface of the fibrous structure 18 at a greater level by weight than within the fibrous structure.

In another example, the surface treating composition and/or lotion composition may be present within the fibrous structure at a greater level by weight than on the surface of the fibrous structure 18.

The surface area coverage of the surface treating composition on the surface of the fibrous structure may be greater than about 10% and/or greater than about 30% and/or greater than about 50% to about 100% and/or to about 90% and/or to about 85%.

The surface area coverage of the lotion composition on the surface of the fibrous structure may be may be greater than about 1% and/or greater than about 5% and/or greater than about 10% and/or greater than about 20% to about 99% and/or to about 90% and/or to about 75% and/or to about 50%.

In one example, the surface area of the fibrous structure and/or sanitary tissue product comprises greater than about 10% and/or greater than about 20% and/or greater than about 50% and/or greater than about 70% and/or greater than about 80% and/or greater than about 90% of the surface treating composition and from 0 to about 90% and/or from 0 to about 80% and/or from 0 to about 50% and/or from 0 to about 30% and/or from 0 to about 20% and/or from 0 to about 10% of the lotion composition. When the surface area of the surface of the fibrous structure and/or sanitary tissue product comprises 0% of the lotion composition, then the lotion may be within the fibrous structure and/or within the sanitary tissue product, such as between two plies of the sanitary tissue product.

In another example, the surface area of the user contacting surface comprises from about 20% to about 97% and/or from about 50% to about 97% and/or from about 80% to about 97% of the surface treating composition and from about 3% to about 80% and/or from about 3% to about 50% and/or from about 3% to about 20% and/or from about 3% to about 15% of the lotion composition.

Surface area coverage of the fibrous structure and/or sanitary tissue product may be determined by the Surface Area Coverage Test Method described herein.

Each region may, within itself, exhibit differential concentrations of their respective compositions and/or differential elevations (protrusions from the surface of the fibrous structure) of their respective compositions

The user contacting surface area may comprise from greater than about 10% and/or greater than about 30% and/or greater than about 50% to about 100% and/or to about 90% and/or to about 85% of the surface treating composition and/or greater than about 1% and/or greater than about 5% and/or greater than about 10% and/or greater than about 20% to about 99% and/or to about 90% and/or to about 75% and/or to about 50% of the lotion composition.

The combination of the surface treating composition and lotion composition in the user contacting surface exhibits softness greater than a user contacting surface comprising either the surface treating composition or lotion composition alone.

The user contacting surface may be planar or may have protrusions of either the surface treating composition and/or lotion composition such that the user contacting surface exhibits differential elevations.

In another example, the user contacting surface may comprise areas of greater concentration and/or greater elevation of the lotion composition, areas of less concentration and/or lesser elevation of the lotion composition, and areas of the surface treating composition.

The surface treating composition and the lotion composition may comprise one or more similar and/or identical ingredients so long as the user contacting surface comprises a first region comprising a different composition (at least one ingredient differs in the composition) than a composition present in a second region.

Nonlimiting types of fibrous structures according to the present invention include conventionally felt-pressed fibrous structures; pattern densified fibrous structures; and high-bulk, uncompacted fibrous structures. The fibrous structures may be of a homogeneous or multilayered (two or three or more layers) construction; and the sanitary tissue products made therefrom may be of a single-ply or multi-ply construction.

The fibrous structures may be post-processed, such as by embossing and/or calendaring and/or folding and/or printing images thereon.

The fibrous structures may be through-air-dried fibrous structures or conventionally dried fibrous structures.

The fibrous structures may be creped or uncreped.

The fibrous structures and/or sanitary tissue products of the present invention may exhibit a basis weight of between about 10 g/m² to about 120 g/m² and/or from about 12 g/m² to about 80 g/m² and/or from about 14 g/m² to about 65 g/m².

The fibrous structures and/or sanitary tissue products of the present invention may exhibit a total dry tensile strength of greater than about 59 g/cm (150 g/in) and/or from about 78 g/cm (200 g/in) and/or from about 98 g/cm (250 g/in) to about 1182 g/cm (3000 g/in) and/or to about 984 g/cm (2500 g/in) and/or to about 787 g/cm (2000 g/in) and/or to about 394 g/cm (1000 g/in) and/or to about 335 g/cm (850 g/in).

The fibrous structure and/or sanitary tissue products of the present invention may exhibit a density of less than about 0.60 g/cm³ and/or less than about 0.30 g/cm³ and/or less than about 0.20 g/cm³ and/or less than about 0.10 g/cm³ and/or less than about 0.07 g/cm³ and/or less than about 0.05 g/cm³ and/or from about 0.01 g/cm³ to about 0.20 g/cm³ and/or from about 0.02 g/cm³ to about 0.10 g/cm³.

The fibrous structures and/or sanitary tissue products of the present invention may exhibit an average lint value of greater than about 0.1 and/or greater than about 0.5 and/or greater than about 1.0 and/or greater than about 1.5 and/or greater than about 2.0 and/or greater than about 3.0 to about 20 and/or to about 15 and/or to about 13 and/or to about 10 and/or to about 8.

Surface Treating Composition

A surface treating composition, for purposes of the present invention, is a composition that improves the tactile sensation of a surface of a fibrous structure perceived by a user whom holds a fibrous structure and/or sanitary tissue product comprising the fibrous structure and rubs it across the user's skin. Such tactile perceivable softness can be characterized by, but is not limited to, friction, flexibility, and smoothness, as well as subjective descriptors, such as a feeling like lubricious, velvet, silk or flannel.

The surface treating composition may or may not be transferable. Typically, it is substantially non-transferable.

The surface treating composition may increase or decrease the surface friction of the surface of the fibrous structure, especially the user contacting surface of the fibrous structure. Typically, the surface treating composition will reduce the surface friction of the surface of the fibrous structure compared to a surface of the fibrous structure without such surface treating composition.

The surface treating composition may have a wettability tension less than or equal to the surface tension of the lotion composition so as to minimize the spreading of the lotion composition that comes into contact with the surface treating composition.

The surface treating composition comprises a surface treating agent. The surface treating composition during application to the fibrous structure may comprise at least about 0.1% and/or at least 0.5% and/or at least about 1% and/or at least about 3% and/or at least about 5% to about 90% and/or to about 80% and/or to about 70% and/or to about 50% and/or to about 40% by weight of the surface treating agent. In one example, the surface treating composition comprises from about 5% to about 40% by weight of the surface treating agent.

The surface treating composition present on the fibrous structure and/or sanitary tissue product comprising the fibrous structure of the present invention may comprise at least about 0.01% and/or at least about 0.05% and/or at least about 0.1% of total basis weight of the surface treating agent. In one example, the fibrous structure and/or sanitary tissue product may comprise from about 0.01% to about 20% and/or from about 0.05% to about 15% and/or from about 0.1% to about 10% and/or from about 0.01% to about 5% and/or from about 0.1% to about 2% of total basis weight of the surface treating composition.

In one example, the surface treating composition of the present invention is a microemulsion of a surface treating agent (for example an aminofunctional polydimethylsiloxane) in water. In such an example, the concentration of the surface treating agent within the surface treating composition may be from about 3% to about 60% and/or from about 4% to about 50% and/or from about 5% to about 40%. Nonlimiting examples of such microemulsions are commercially available from Wacker Chemie, Dow Coming and/or General Electric Silicones.

Nonlimiting examples of suitable surface treating agents can be selected from the group consisting of: polymers such as polyethylene and derivatives thereof, hydrocarbons, waxes, oils, silicones (polysiloxanes), quaternary ammonium compounds, fluorocarbons, substituted C₁₀-C₂₂ alkanes, substituted C₁₀-C₂₂ alkenes, in particular derivatives of fatty alcohols and fatty acids(such as fatty acid amides, fatty acid condensates and fatty alcohol condensates), polyols, derivatives of polyols (such as esters and ethers), sugar derivatives (such as ethers and esters), polyglycols (such as polyethyleneglycol) and mixtures thereof.

Nonlimiting examples of suitable waxes may be selected from the group consisting of: paraffin, polyethylene waxes, beeswax and mixtures thereof.

Nonlimiting examples of suitable oils may be selected from the group consisting of: mineral oil, silicone oil, silicone gels, petrolatum and mixtures thereof.

Nonlimiting examples of suitable silicones (polysiloxanes) may be selected from the group consisting of: polydimethylsiloxanes, aminosilicones, cationic silicones, quaternary silicones, silicone betaines and mixtures thereof.

Nonlimiting examples of suitable polysiloxanes and/or monomeric/oligomeric units may be selected from the compounds having monomeric siloxane units of the following structure: 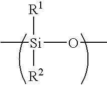
wherein, R¹ and R2, for each independent siloxane monomeric unit can each independently be hydrogen or any alkyl, aryl, alkenyl, alkaryl, arakyl, cycloalkyl, halogenated hydrocarbon, or other radical. Any of such radical can be substituted or unsubstituted. R¹ and R² radicals of any particular monomeric unit may differ from the corresponding functionalities of the next adjoining monomeric unit. Additionally, the polysiloxane can be either a straight chain, a branched chain or have a cyclic structure. The radicals R¹ and R² can additionally independently be other silaceous functionalities such as, but not limited to siloxanes, polysiloxanes, silanes, and polysilanes. The radicals R¹ and R² may contain any of a variety of organic functionalities including, for example, alcohol, carboxylic acid, phenyl, and amine functionalities. The end groups can be reactive (alkoxy or hydroxyl) or nonreactive (trimethylsiloxy). The polymer can be branched or unbranched.

Exemplary alkyl radicals are methyl, ethyl, propyl, butyl, pentyl, hexyl, octyl, decyl, octadecyl, and the like. Exemplary alkenyl radicals are vinyl, allyl, and the like. Exemplary aryl radicals are phenyl, diphenyl, naphthyl, and the like. Exemplary alkaryl radicals are toyl, xylyl, ethylphenyl, and the like. Exemplary aralkyl radicals are benzyl, alpha-phenylethyl, beta-phenylethyl, alpha-phenylbutyl, and the like. Exemplary cycloalkyl radicals are cyclobutyl, cyclopentyl, cyclohexyl, and the like. Exemplary halogenated hydrocarbon radicals are chloromethyl, bromoethyl, tetrafluorethyl, fluorethyl, trifluorethyl, trifluorotloyl, hexafluoroxylyl, and the like.

Viscosity of polysiloxanes useful for this invention may vary as widely as the viscosity of polysiloxanes in general vary, so long as the polysiloxane can be rendered into a form which can be applied to the fibrous structures herein. This includes, but is not limited to, viscosity as low as about 25 centistokes to about 20,000,000 centistokes or even higher.

Nonlimiting examples of suitable quaternary ammonium compounds may be selected from compounds having the formula: 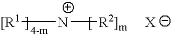
wherein:
m is 1 to 3; each R¹ is independently a C₁-C₆ alkyl group, hydroxyalkyl group, hydrocarbyl or substituted hydrocarbyl group, alkoxylated group, benzyl group, or mixtures thereof; each R² is independently a C₁₄-C₂₂ alkyl group, hydroxyalkyl group, hydrocarbyl or substituted hydrocarbyl group, alkoxylated group, benzyl group, or mixtures thereof; and X⁻ is any quaternary ammonium-compatible anion.

In one example, each R¹ is methyl and X⁻ is chloride or methyl sulfate and each R² is independently C₁₆-C₁₈ alkyl or alkenyl. Each R² may be independently straight-chain C₁₈ alkyl or alkenyl.

In another example, the quaternary ammonium compounds may be mono or diester variations having the formula:
(R¹)₄₋ₘ—N+—[(CH₂)ₙ—Y—R³]ₘX⁻
wherein:
Y is —O—(O)C—, or —C(O)—O—, or —NH—C(O)—, or —C(O)—NH—; m is 1 to 3; n is 0 to 4; each R¹ is independently a C₁-C₆ alkyl group, hydroxyalkyl group, hydrocarbyl or substituted hydrocarbyl group, alkoxylated group, benzyl group, or mixtures thereof; each R³ is independently a C₁₃-C₂₁ alkyl group, hydroxyalkyl group, hydrocarbyl or substituted hydrocarbyl group, alkoxylated group, benzyl group, or mixtures thereof, and X⁻ is any quaternary ammonium-compatible anion.

In one example, Y is —O—(O)C—, or —C(O)—O—; m=2; and n=2, each R¹ is independently a C₁-C₃, alkyl group, each R³ is independently C₁₃-C₁₇ alkyl and/or alkenyl. In another example each R¹ is methyl and each R³ is independently a straight chain C ₁₅-C₁₇ alkyl and/or alkenyl.

In another example, the quaternary ammonium compound may be an imidazolinium compound, such as an imidazolinium salt.

As mentioned above, X⁻ can be any quaternary ammonium-compatible anion, for example, acetate, chloride, bromide, methyl sulfate, formate, sulfate, nitrate and the like can also be used in the present invention. In one example, X⁻ is chloride or methyl sulfate.

The surface treating composition may comprise additional ingredients such as a vehicle as described herein below which may not be present on the fibrous structure and/or sanitary tissue product comprising such fibrous structure. In one example, the surface treating composition may comprise a surface treating agent and a vehicle such as water to facilitate the application of the surface treating agent onto the surface of the fibrous structure.

Lotion Composition

The lotion composition may comprise oils and/or emollients and/or waxes and/or immobilizing agents. In one example, the lotion composition comprises from about 10% to about 90% of an oil and/or liquid emollient and from about 10% to about 50% of immobilizing agent and/or from about 0% to about 60% of petrolatum and optionally the balance of a vehicle.

The lotion compositions may be heterogeneous. They may contain solids, gel structures, polymeric material, a multiplicity of phases (such as oily and water phase) and/or emulsified components. It may be difficult to determine precisely the melting temperature of the lotion composition, i.e. difficult to determine the temperature of transition between the liquid form, the quasi-liquid from, the quasi-solid form and the solid form. The terms melting temperature, melting point, transition point and transition temperature are used interchangeably in this document and have the same meaning.

The lotion compositions may be semi-solid, of high viscosity so they do not substantially flow without activation during the life of the product or gel structures.

The lotion compositions may be shear thinning and/or they may strongly change their viscosity around skin temperature to allow for transfer and easy spreading on a user's skin.

The lotion compositions may be in the form of emulsions and/or dispersions.

In one example of a lotion composition, the lotion composition has a water content of less than about 20% and/or less than 10% and/or less than about 5% or less than about 0.5%.

In another example, the lotion composition may have a solids content of at least about 15% and/or at least about 25% and/or at least about 30% and/or at least about 40% to about 100% and/or to about 95% and/or to about 90% and/or to about 80%.

Nonlimiting examples of suitable oils and/or emollients include glycols (such as propylene glycol and/or glycerine), polyglycols (such as triethylene glycol), petrolatum, fatty acids, fatty alcohols, fatty alcohol ethoxylates, fatty alcohol esters and fatty alcohol ethers, fatty acid ethoxylates, fatty acid amides and fatty acid esters, hydrocarbon oils (such as mineral oil), squalane, fluorinated emollients, silicone oil (such as dimethicone) and mixtures thereof.

Suitable fatty acid ester type emollients include those derived from C₁₂-C₂₈ fatty acids, such as C₁₆-C₂₂ saturated fatty acids, and short chain (C₁-C₈ and/or C₁-C₃) monohydric alcohols. Representative examples of such esters include methyl palmitate, methyl stearate, isopropyl laurate, isopropyl myristate, isopropyl palmitate, and ethylhexyl palmitate. Suitable fatty acid ester emollients can also be derived from esters of longer chain fatty alcohols (C₁₂-C₂₈ and/or C₁₂-C₁₆) and shorter chain fatty acids e.g., lactic acid, such as lauryl lactate and cetyl lactate. Suitable alkyl ethoxylate type emollients include C₁₂-C₁₈ fatty alcohol ethoxylates having an average of from about 3 to about 30 and/or from about 4 to about 23 oxyethylene units. Nonlimiting examples of such alkyl ethoxylates include laureth-3 (a lauryl ethoxylate having an average of 3 oxyethylene units), laureth-23 (a lauryl ethoxylate having an average of 23 oxyethylene units), ceteth-10 (acetyl ethoxylate having an average of 10 oxyethylene units) and steareth-10 (a stearyl ethoxylate having an average of 10 oxyethylene units). These alkyl ethoxylate emollients can be used in combination with other emollients, such as petroleum-based emollients, such as petrolatum, at a weight ratio of alkyl ethoxylate emollient to petroleum-based emollient of from about 1:1 to about 1:3 and/or from about 1:1.5 to about 1:2.5.

Immobilizing agents include agents that are may prevent migration of the emollient into the fibrous structure such that the emollient remain primarily on the surface of the fibrous structure and/or sanitary tissue product and/or on the surface treating composition on a surface of the fibrous structure and/or sanitary tissue product and facilitate transfer of the lotion composition to a user's skin. Immobilizing agents may function as viscosity increasing agents and/or gelling agents.

Nonlimiting examples of suitable immobilizing agents include waxes (such as ceresin wax, ozokerite, microcrystalline wax, petroleum waxes, fisher tropsh waxes, silicone waxes, paraffin waxes), fatty alcohols (such as cetyl and/or stearyl alcohol), fatty acids and their salts (such as metal salts of stearic acid), mono and polyhydroxy fatty acid esters, mono and polyhydroxy fatty acid amides, silica and silica derivatives, gelling agents, thickeners and mixtures thereof.

In one example, the lotion composition comprises at least one immobilizing agent and at least one emollient.

In one example, the lotion composition comprises a sucrose ester of a fatty acid.

The lotion composition may be added to a fibrous structure at any point during the papermaking and/or converting process. In one example, the lotion composition is added to the fibrous structure during the converting process.

The lotion composition may be a transferable lotion composition. A transferable lotion composition comprises at least one component that is capable of being transferred to an opposing surface such as a user's skin upon use. In one example, at least 0.1% of the transferable lotion present on the user contacting surface transfers to the user's skin during use. The amount of transferable composition that transfers to a user's skin during use can be determined by known methods such as by tape stripping the skin 3 times, after use of the fibrous structure and/or sanitary tissue product by the user, with Tegaderm Tapes, available from 3M, and analyzing the tapes for the transferable composition or a component within the transferable composition assuming all components of the transferable composition transfer equally.

Other optional components that may be included in the lotion composition include vehicles, perfumes, especially long lasting and/or enduring perfumes, antibacterial actives, antiviral actives, disinfectants, pharmaceutical actives, film formers, deodorants, opacifiers, astringents, solvents, cooling sensate agents, and the like. Particular examples of lotion composition components include camphor, thymol, menthol, chamomile extracts, aloe vera, calendula officinalis, alpha bisalbolol, Vitamin E, Vitamin E acetate.

In one example of the lotion composition of the present invention, the lotion composition has a melting point greater than about 35° C. For example, the lotion composition has to be subjected to a temperature of greater than about 35° C. before a substantial amount (for example, greater than 30% and/or greater than 40% and/or greater than 50% and/or greater than 60%) of the lotion composition melts. This can be expressed as:
(1) ΔH²/ΔH¹ is equal to or larger than about 1 and/or equal to or larger than about 4 and/or equal to or larger than about 9; and/or
(2) ΔH² is equal to or larger than about 30 J/g, 40 J/g and/or equal to or larger than about 60 J/g (especially if ΔH¹ is 0)
   wherein: ΔH¹ is the energy required to raise the temperature of the lotion composition from 15° C. to 35° C.; ΔH² is the energy required to raise the temperature of the lotion composition from 35° C. to the temperature where the lotion composition is fully liquid or where no more melting occurs below 100° C. in the case the lotion composition contains components only melting above 100° C.

ΔH is measured by DSC technique using standard parameters known to the one skilled in the art. DSC data are obtained using a Thwing Albert DSC 2920 Instrument, calibrated with an indium metal standard with a melting onset temperature of 156.6° C. and a heat of melting of 6.80 calories per gram, as reported in the literature. The sample is first heated to 100° C. at a rate of 10° C./min, equilibrated for 5 minutes at 100° C., cooled down to −30° C. at a rate of −2.5° C./min, equilibrated at −30° C. for 5 minutes and then finally heated from −30° C. to +100° C. at a rate of 2.5° C./min to evaluate the melt behaviour. For determination of ΔH¹ and ΔH² the final heating ramp is used. ΔH¹ is the area between the DSC curve and the baseline between 15° C. and 35° C. and ΔH² is the area between the DSC curve and the baseline between 35° C. and the temperature where the lotion composition is fully liquid or where no more melting occurs below 100° C. in the case the lotion composition contains components only melting above 100° C. By way of example, a lotion composition of the present invention that comprises about 40% Stearylalcohol, about 30% Mineral oil and about 30% Petrolatum has a value of ΔH²/ΔH¹>9 and a value of ΔH²>60 J/g.

In one example, the lotion composition is present on the surface of the fibrous structure and/or sanitary tissue product and/or on the surface treating composition present on the surface of the fibrous structure and/or sanitary tissue product at a level of at least about 0.5 g/m² and/or at least about 1.0 g/m² and/or at least about 1.5 g/m² per user contacting surface. In another example, the lotion composition is present on the surface of the fibrous structure and/or sanitary tissue product and/or on the surface treating composition present on the surface of the fibrous structure and/or sanitary tissue product at a level of from about 0.5 g/m² and/or from about 1.0 g/m² and/or from about 1.5 g/m² to about 10 g/m² and/or to about 8 g/m² and/or to about 6 g/m² per user contacting surface.

Vehicle

As used herein a “vehicle” is a material that can be used to dilute and/or emulsify agents forming the surface treating composition and/or lotion composition to form a dispersion/emulsion. A vehicle may be present in the surface treating composition and/or lotion composition, especially during application of the surface treating composition and/or to the fibrous structure. A vehicle may dissolve a component (true solution or micellar solution) or a component may be dispersed throughout the vehicle (dispersion or emulsion). The vehicle of a suspension or emulsion is typically the continuous phase thereof. That is, other components of the dispersion or emulsion are dispersed on a molecular level or as discrete particles throughout the vehicle.

Suitable materials for use as the vehicle of the present invention include hydroxyl functional liquids, including but not limited to water. In one example, the lotion composition comprises less than about 20% and/or less than about 10% and/or less than about 5% and/or less than about 0.5% w/w of a vehicle, such as water. In one example, the surface treating composition comprises greater than about 50% and/or greater than about 70% and/or greater than about 85% and/or greater than about 95% and/or greater than about 98% w/w of a vehicle, such as water.

Process Aids

Process aids may also be used in the lotion compositions of the present invention. Nonlimiting examples of suitable process aids include brighteners, such as TINOPAL CBS-X®, obtainable from CIBA-GEIGY of Greensboro, N.C.

### NONLIMITING EXAMPLES OF LOTION COMPOSITIONS

### Example 1 of Lotion Composition

| | |
|---|---|
| Stearyl Alcohol CO1897* | 40% w/w |
| Petrolatum Snowwhite V28EP** | 30% w/w |
| Mineral oil Carnation** | 30% w/w |

*Available from Procter&Gamble Chemicals, Cincinnati, USA
**Available from Crompton Corporation

The lotion composition has a melting point of about 51° C. and a melt viscosity at 56° C. of about 17 m*Pas measured at a shear rate of 0.1 l/s. The mineral oil used in this formulation has a viscosity of about 21 mPa*s at 20° C. The lotion composition can be applied to one or both surfaces of the fibrous structure at total add-on levels of 3.6 g/m², 4.2 g/m², 6 g/m², 7.2 g/m², 8.4 g/m² and 11.4 g/m².

Processes for Treating Fibrous Structures and/or Sanitary Tissue Products

a. Surface Treating Composition:

Any contact or contact free application suitable for applying the surface treating composition, such as spraying, dipping, padding, printing, slot extruding, rotogravure printing, flexographic printing, offset printing, screen printing, mask or stencil application process and mixtures thereof can be used to apply the surface treating composition to the fibrous structure and/or sanitary tissue product and/or to the lotion composition present on a surface of the fibrous structure and/or sanitary tissue product. Surface treating compositions can be applied to the fibrous structure and/or sanitary tissue product before, concurrently, or after the lotion composition application to the fibrous structure and/or sanitary tissue product. The surface treating composition can be applied during papermaking and/or converting, especially if applied to the outside layer of a layered fibrous structure and/or sanitary tissue product comprising such layered fibrous structure.

In one example, the surface treating composition is applied by an application process that provides a relatively high surface area coverage on the surface of the fibrous structure and/or sanitary tissue product. Examples of such suitable application process include, but are not limited to, printing, slot extruding and/or spraying with fine particles (although spraying has disadvantage of producing aerosoles if high area coverage is to be achieved).

b. Lotion Composition:

Any contact or contact free application suitable for applying the lotion composition, such as spraying, dipping, padding, printing, slot extruding, rotogravure printing, flexographic printing, offset printing, screen printing, mask or stencil application process and mixtures thereof can be used to apply the lotion composition to the fibrous structure and/or sanitary tissue product and/or surface treating composition present on the surface of the fibrous structure and/or sanitary tissue product. The lotion composition can be applied to the fibrous structure and/or sanitary tissue product before, concurrently, and/or after the surface treating composition application to the fibrous structure and/or sanitary tissue product. In one example, the lotion composition is applied to the surface treating composition present on the surface of the fibrous structure and/or sanitary tissue product.

In one example, the lotion composition is applied by an application process that provides a relatively low surface area coverage on the surface of the fibrous structure and/or sanitary tissue product and/or on the surface treating composition present on the surface of the fibrous structure and/or sanitary tissue product such that regions of surface treating composition and regions of lotion composition produce the user contacting surface. Example of such suitable application processes include, but are not limited to, spraying, especially spraying with rotating discs, printing, slot extruding in stripes and/or other patterns.

In one example, the surface treating composition may be added to a fiber furnish that will form an external layer of a multilayer fibrous structure. The lotion composition may be applied to the surface formed by the external layer of the multilayer fibrous structure.

In one example, the surface treating composition is applied to the surface of the fibrous structure during the fibrous structure making process, such as before and/or after drying the fibrous structure. The lotion composition may then be applied to the surface treating composition on the surface of the fibrous structure during the converting process.

In one example, the surface treating composition contains less than about 5% and/or less than about 3% and/or less than about 1% and/or less than about 0.5% moisture at the time the lotion composition is applied to it.

### TREATED FIBROUS STRUCTURE EXAMPLES

### Fibrous Structure

### Example 1

A first fibrous structure is a conventional wet pressed, homogeneous, dry creped fibrous structure with a basis weight of about 15.4 g/m². The fibrous structure has a composition of about 40% Northern Softwood Kraft and 60% Eucalyptus. Four plies of the fibrous structure are combined together in an off line combining operation to produce a sanitary tissue product. The 4-ply sanitary tissue product has a basis weight of about 60 g/m², a thickness of about 0.3 mm, a machine direction strength of about 1280 g/in, a cross direction strength of about 610 g/in, and a wet burst of about 200 g. It contains a wet strength agent and a dry strength agent.

### Fibrous Structure

### Example 2

A second fibrous structure is a conventional wet pressed, layered, dry creped fibrous structure with a basis weight of about 14.6 g/m². The outer layer contains about 100% Eucalyptus fiber whereas the inner layer is composed of a furnish mix of about 85% Northern Softwood Kraft, 10% CTMP and about 5% Eucalyptus fiber. Both layers are of about equal basis weight (symmetrical layer split). Four plies of the fibrous structure are combined together in an off line combining operation to form a sanitary tissue product such that the Eucalyptus layer is present on the two outer surfaces of the combined 4-ply sanitary tissue product. The 4-ply sanitary tissue product has a basis weight of about 60 g/m², a thickness of about 0.3 mm, a machine direction strength of about 1180 g/in, a cross direction strength of about 560 g/in, and a wet burst of about 200 g. It contains a wet strength agent and a dry strength agent.

### Fibrous Structure

### Example 3

A third fibrous structure is formed from an aqueous slurry of Northern Softwood Kraft (NSK) of about 3% consistency made up using a conventional pulper and passed through a stock pipe toward the headbox of the Fourdrinier. A 1% dispersion of Hercules' Kymene 557 LX is prepared and is added to the NSK stock pipe at a rate sufficient to deliver about 0.8% Kymene 557 LX based on the dry weight of the ultimately resulting sanitary tissue product. The absorption of the permanent wet strength resin is enhanced by passing the treated slurry through an in-line mixer. An aqueous solution of Carboxymethyl cellulose (CMC) dissolved in water and diluted to a solution strength of 1% is added next to the NSK stock pipe after the in-line mixer at a rate of about 0.1% CMC by weight based on the dry weight of the ultimately resulting sanitary tissue product. The aqueous slurry of NSK fibers passes through a centrifugal stock pump to aid in distributing the CMC. An aqueous dispersion of DiTallow DiMethyl Ammonium Methyl Sulfate (DTDMAMS) (170° F.) at a concentration of 1% by weight is added to the NSK stock pipe at a rate of about 0.1% by weight DTDMAMS based on the dry weight of the ultimately resulting sanitary tissue product. An aqueous slurry of eucalyptus bleached kraft fibrous pulp fibers (from Aracruz—Brazil) of about 1.5% by weight is made up using a conventional repulper and is passed through a stock pipe toward the headbox of the Fourdrinier. This Eucalyptus furnish joins the NSK slurry at the fan pump where both are diluted with white water to about 0.2% consistency. An aqueous slurry of eucalyptus bleached kraft fibrous pulp fibers (from Aracruz—Brazil) of about 3% by weight is made up using a conventional repulper. The Eucalyptus slurry passes to the second fan pump where it is diluted with white water to a consistency of about 0.2%. The slurries of NSK/eucalyptus and eucalyptus are directed into a multi-channeled headbox suitably equipped with layering leaves to maintain the streams as separate layers until discharged onto a traveling Fourdrinier wire. A three-chambered headbox is used. The eucalyptus slurry containing 48% of the dry weight of the ultimate sanitary fibrous structure is directed to the chamber leading to the layer in contact with the wire, while the NSK/eucalyptus slurry comprising 52% (27-35% NSK and 17-25% eucalyptus) of the dry weight of the ultimate paper is directed to the chamber leading to the center and inside layer. The NSK/eucalyptus and eucalyptus slurries are combined at the discharge of the headbox into a composite slurry. The composite slurry is discharged onto the traveling Fourdrinier wire and is dewatered assisted by a deflector and vacuum boxes. The embryonic wet fibrous structure is transferred from the Fourdrinier wire, at a fiber consistency of about 17% by weight at the point of transfer, to a patterned drying fabric. The drying fabric is designed to yield a pattern-densified tissue with discontinuous low-density deflected areas arranged within a continuous network of high density (knuckle) areas. This drying fabric is formed by casting an impervious resin surface onto a fiber mesh supporting fabric. The supporting fabric is a 48×52 filament, dual layer mesh. The thickness of the resin cast is about 8 mil above the supporting fabric. The knuckle area is about 35-50% and the open cells remain at a frequency of about 68-562 per square inch. Further de-watering is accomplished by vacuum assisted drainage until the fibrous structure has a fiber consistency of about 23-27%. While remaining in contact with the patterned forming fabric, the patterned fibrous structure is pre-dried by air blown through to a fiber consistency of about 60% by weight. The semi-dry fibrous structure is then adhered to the surface of a Yankee dryer with a sprayed creping adhesive comprising a 0.250% aqueous solution of polyvinyl alcohol. The creping adhesive is delivered to the Yankee surface at a rate of 0.1% adhesive solids based on the dry weight of the fibrous structure. The fiber consistency is increased to about 98% before the fibrous structure is dry creped from the Yankee with a doctor blade. After the doctor blade, the fibrous structure is calendared across all its width with a steel to rubber calendar roll operating at a loading of 300-500 psi. The resulting tissue has a basis weight of about 20-25 g/m2; a 1-ply total dry tensile between 250 and 370 g/in, a 1-ply wet burst between 35 and 65 gr/in and a 2-ply caliper of about 0.015-0.020 inches. The resulting tissue is then combined with a like sheet to form a two-ply, creped, pattern-densified tissue so that the eucalyptus fibers face the outside and it is subjected to calendaring between two smooth steel calendar rolls. The product is then ply-bonded using a mechanical plybond wheel to ensure that both plies stay together. The resulting two-ply tissue has a) a total basis weight of about 39-50 g/m2; b) a 2-ply total dry tensile between 450 and 700 gr/in; c) a 2-ply wet burst between 100 and 130 g/in; d)a 4-ply caliper of about 0.51 and 0.89 mm.

The fibrous structures described above can be used in combination with any of the treatment processes and lotion compositions described below.

### Converting of the Fibrous Structures of Examples 1-3

The combined parent roll is subsequently converted into a sanitary tissue product. The multi-ply parent roll is unwound and subjected to calandering between two smooth steel calender rolls followed by high pressure embossing to achieve ply bonding. The majority of the fibrous structure remains unaffected by the high pressure embossing. The surface treating composition and the lotion composition are then applied to the fibrous structure as described in detail below. Finally the tissue was cut in machine direction, followed by cutting in cross direction into sheets of approximately 21 cm×21 cm, folded, stacked into stacks of 9 sheets and packed into individual pocket packs.

### Applying Surface Treating Composition to Fibrous Structures of Examples 1-3

Directly following the ply bond operation, the surface treating composition is printed onto the surface of the 4-ply fibrous structure and/or sanitary tissue product using a roto-gravure printing process. About 1.5 g/m² of the surface treatment composition is transferred to each side of the 4-ply product.

The printing station consists of two engraved anilox rolls facing each other in a horizontal arrangement and forming a gap in between through which the fibrous structure and/or sanitary tissue product is run. The geometry is arranged in a way that the rolls touch the fibrous structure and/or sanitary tissue product and transfer lotion composition macroscopically uniformly onto both surfaces of the 4-ply fibrous structure and/or sanitary tissue product but the fibrous structure and/or sanitary tissue product does not wrap any of the two anilox rolls. The anilox rolls are engraved to a cell volume of about 3 ml per square meter and about 100 cells per square centimeter, and supplied with lotion composition from a closed supply chamber designed to fill the engraved volume with lotion composition. The gap between the two rolls is adjusted to achieve the target add-on level. Surface coverage of the surface treatment composition was substantially 100% and homogenious. Surface coverage can, e.g. be tested using a surface treatment composition that has 0.01% Tinopal CBS-X added, a fluorescent dye, available from Ciba Speciality Chemicals, Basel, Switzerland.

Samples have also been made using an identical application system as described below for the application of lotion composition. The equipment was operated at ambient temperature at a disc speed of about 4000 rpm. Surface coverage of this application is lower than using a printing process as described above. While still within the scope of this invention, this process is therefore less preferred.

### Applying Lotion Composition to Fibrous Structures of Examples 1-3

Directly following the surface treating composition, the lotion composition is applied to the fibrous structure and/or sanitary tissue product. The fibrous structure and/or sanitary tissue product span between the two operations was about 5 meter. A commercially available rotary spray application system RFT-Compact-III with applicator heads for the tissue and textile industry (available from Weitmann&Konrad GmbH & Co KG, Leinfelden Echterdingen, Germany) was modified to be used to practice the present invention. The application head is equipped with 5 sets of rotary disks (type 1/1) and has an effective application width of 448 mm. The housing of the application head was replaced with water heated walls on the top, the bottom and the rear side of the application head. The whole unit was then insulated towards the outside. Two of these modified application heads were used, installed facing each other so that both sides of a fibrous structure and/or sanitary tissue product can be treated simultaneously. Heating units with an integrated pump (Type W60/10-12/40, available from Kelviplast GmbH, Germany) are used to supply the application units with water of the desired temperature. In particular, the design of the heating elements was chosen so that the temperature inside the application head is within +/−2° C. from the target temperature. The lotion composition infeed of the application heads are connected through a heat traced piping system to a heated pump that is connected through heat traced piping to a heated 100 liter tank that holds the melted lotion composition. The return lines of the applicator feed back into the heated tank. A heated flow meter was installed in the lotion composition supply line between pump and application heads. The flow meter (Promass 63M, available from Endress & Hauser, Switzerland) was connected to the control unit of the RFT-Compact-III system that was then used to control the lotion composition pump (Gear pump of type Labu Brox) to deliver the desired lotion composition flow to the application heads.

No changes are made to the setup, shape and dimensions of the rotating surfaces in the commercially available application head. Each set of rotating surfaces consisted of 2 rotating discs stacked on top of each other. The lotion composition supply to the two rotating surfaces of each stack is equally split. The discs have a diameter of about 98 mm. The five individual stacks of rotating surfaces are spaced apart by about 112 mm. The first, third and fifth set of rotating surfaces is installed vertically shifted versus the second and fourth stack of rotating surfaces to avoid interference between the horizontally overlapping streams of droplets. The sets of rotating surfaces are commercially available from Weitmann & Konrad GmbH & Co, Germany (type 1/1, Art. No. 618996 [upper set] and 618997 [lower set]). The applicator is operated horizontally and with a distance of about 154 mm between the fibrous structure and/or sanitary tissue product and the center of the disks. The fibrous structure and/or sanitary tissue product is run vertically from top to bottom between the two application heads. Controlled by the windows in the housing between the rotating surfaces and the fibrous structure and/or sanitary tissue product, each stack of rotating surfaces covers a cross direction width of about 224 mm on the fibrous structure and/or sanitary tissue product with the exception of the two outer stacks of rotating surfaces of the applicator which only cover 112 mm each. At each position the streams of two stacks of rotating surfaces are overlapping. Even distribution to the individual stacks of discs was achieved with throttles of 1 mm diameter, installed between the infeeds to the rotary discs and the central supply pipe of the applicator. The lotion composition temperature is controlled to a determined value through the heating of the tank, the piping and the temperature in the application heads to the desired value. The flow rate is adjusted to achieve the desired add-on level of the fibrous structure. During application, the fibrous structure and/or sanitary tissue product is typically kept at room temperature. Some samples were made where the fibrous structure was cooled or heated prior to application of the lotion composition. The lotion composition almost instantaneously solidifies after impacting the fibrous structure and/or sanitary tissue product. Samples with add-on levels of 1.5 g/m², 2.3 g/m², 3 g/m² and 4.5 g/m² per fibrous structure and/or sanitary tissue product side are made.

The surface area coverage of the lotion composition is about 15% for the sample made with 3 g/m² lotion application per fibrous structure and/or sanitary tissue product side, a disc speed of about 2500 rpm, and a lotion composition temperature in the applicator of about 56° C.

### Process Conditions for Treating of Fibrous Structures of Examples 1-3

The rotating surfaces are operated at 2500 rpm for the samples described below but additional samples are made at speeds between 200 rpm and 5000 rpm.

The lotion composition is usually maintained at a temperature of about 5-10° C. above the melting point, for the lotion compositions described below all temperature settings are kept at 56° C. Products are made at temperatures less than 2° C. below and more than 10° C. above the melting point. The fibrous structure and/or sanitary tissue product speed for the examples below is 200 m/min, but samples can be made at fibrous structure and/or sanitary tissue speeds between 10 m/min and 400 m/min.

Test Methods

A. Surface Area Coverage Test Method

The local surface treating composition and/or lotion composition basis weight on the surface of the fibrous structure can be determined by scanning IR/NIR (infrared or near infrared) spectroscopy in transmission mode (absorption spectroscopy) using a Perkin Elmer Spectrum Spotlight 300 instrument in combination with Spotlight software version 1.1.0 B38.

The following procedure is applicable to surface treating compositions and/or lotion compositions comprising a linear hydrocarbon component of repeated —(CH2)— units. Adaptation of the procedure may become necessary if the composition is composed mostly or entirely of other materials. Such adaptations will depend on the composition and will usually be apparent to those skilled in the art.

The measurements are done with samples representative for the tissue. A 5×5 mm sample (or larger) is placed on the sample holder, which is mounted on a XY table and the spectral area used for analysis is scanned at a spatial resolution of 25 μm in both x and y dimension. For the analysis of materials containing linear chains of —CH2— groups the region between 4000 cm-1 and 4500 cm-1 is scanned and the range between 4296 cm-1 (W1) and 4368 cm-1 (W2) is used for analysis. At least 16 scans are taken at a resolution of 1 cm-1. If more than 16 scans are used, care needs to be taken that the sample does not change structure as a result of heating up.

Next, a map of the local basis weight of the sample is generated. The integrated absorption between W2 and W1 and above a sloping linear baseline is determined for each pixel of 25 μm×25 μm using the ChemiMap menu of the software. The baseline is defined by the absorbency at W1 and W2. The two base points option is chosen in the ChemiMap menu of the software and set at W1 and W2. Start and end point of the integration are also set at W1 and W2. The scaling factor is set to a value V1 which is defined as: V1=F*DW where F is the factor described below and DW=W2−W1 is the delta in-wave numbers-between the upper (W2) and the lower (W1) wave number in cm⁻¹.

The scaling with the factor DW transforms the average absorbance above the baseline within the wave number range W1 to W2 into an integrated absorption above the baseline. The factor F translates the integrated absorption into local basis weight in g/m².

The file, which is generated with the ChemiMap command, contains the local basis weight for each pixel of 25 μm×25 μm in area. The file is saved as a text file (.txt format) and also as a bitmap (.bmp format) in 8 bit grey scale format. The text file is imported into EXCEL and the first row and first column are removed (they do not contain image data, but position data). The resulting data are representing the array of pixels of local basis weight in g/m². The maximum (MaxLBW) and minimum (MinLBW) value, as well as the average (AvgLBW) of the whole dataset is calculated in EXCEL.

The bitmap file (.bmp file) is imported into AnalySIS image analysis software for further processing (Analysis Pro version 3.1 (build 508), available from Soft Imaging GmbH, Germany). The imported grey scale file is still in RGB format with all three color channels set equal (in 8 bit resolution). In AnalySIS the file is color separated to extract one of the three identical color channels (red). The resulting file is now scaled from G=0 to G=255, G=0 representing the minimum value (MinLBW) of the original spotlight data and 255 representing the maximum value (MaxLBW) of the original spotlight data. The image is calibrated in x-y by setting the pixel size in x and y dimension to match the original sample. The image is rescaled in z-direction to display the local basis weight values in g/m² but all calculations within AnalySIS have to be made in the G=0 to G=255 scale. The G values can be easily transformed into local basis weight numbers by the following relationship:
LLBW=A*(G+OFFSET), where A=(MaxLBW−MinLBW)/255 and
OFFSET=(255*MinLBW)/(MaxLBW−MinLBW)

The G values can be easily transformed into local lotion basis weight numbers (LLBW) by the following relationship: G=(LLBW/A)−OFFSET

LLBW can be local lotion composition basis weight or local surface treating composition basis weight depending upon what is being measured.

The average value of all local basis weight datapoints above 10 g/m² can be calculated from the EXCEL datafile.

The area of fibrous structure and/or sanitary tissue product affected by the composition is calculated in Analysis by setting a lower threshold at the G value equivalent to 3 g/m² and calculating the area above that threshold. The setting “holes not filled” is used. The areas of the composition is similarly determined by setting the threshold at a G value equivalent to 10 g/m² (10 g/m² equals G=10/A−OFFSET).

If the areas of the composition are defined to have a certain minimum and/or maximum area is set as a filter. The area percentage of composition larger than a certain area is calculated by dividing the area of the composition calculated without area filter, divided by the area of the composition calculated with area filter.

The factor F to convert integrated absorption values into local lotion basis weight values is determined by the following procedure: A representative set of calibration samples of known average basis weight of the composition is scanned in the spectral range used for the analysis as described above and analyzed for integrated peak area between W1 and W2 (4296 cm-1 and 4368 cm-1 for mostly hydrocarbon like materials). The integrated peak area is obtained from the procedure above if the factor F is set equal to 1. The dataset is then imported to EXCEL and the average pixel value of this dataset is calculated. As the factor F was set equal to 1 this value is equal to the mean integrated peak area (AIPA) of the sample in the wave number range W1 to W2. The factor F is then calculated as F=1/slope of a linear least square fit through the origin of the plot of AIPA vs. average composition basis weight of the sample. Calibration samples to determine the factor F can either be prepared or an existing composition-containing sample can be used. If an existing sample is used the composition basis weight can be determined by extraction. An example for such a procedure is given below. Examples for how the factor F is determined by analyzing an existing sample (market product) and by preparing calibration samples is also given. below. It is important, that the absorbency in the wavelength range used for analysis should never exceed about 1 to ensure a linear correlation between the infrared signal and the local composition basis weight

i. Determination of Factor F by Preparing Calibration Samples

Preparation of calibration samples: A suitable piece of the substrate of known area, weight and basis weight is evenly treated with the composition. A suitable type of equipment is a hot wax cartridge spray gun type MK-DUO Line Art. No. 140101, available from MK Heiβwachstechnik GmbH, Aichach, Germany. After the application, the composition is equilibrated in the sheet by placing the sample in an oven at a temperature of about 10° C. above the mp (or at a temperature suitable to allow for sufficient equilibration of the composition in/on the sheet). For relatively low viscosity samples equilibration for about an hour is sufficient. The sample is then cooled down to room temperature and equilibrated for moisture content at 23° C. (+−1° C.) and 50% (+−2%) relative humidity and weighed again. The composition basis weight of that sample [in g/m²] is then calculated as (sample weight after composition treatment [in grams]—sample weight before composition treatment [in grams]) divided by area of the sample [in m²]. The samples are then analyzed by the procedure described above to determine the factor F. Preferably, calibration samples are prepared in a range of composition basis weights that include the range to be measured.

Determination of Factor F for a market product: The basis weight of the sample is determined by a standard procedure. The sample is then analyzed by the procedure described above for the average integrated peak area between 4296 cm-1 and 4368 cm-1. The sample is then extracted by the procedure described below to determine the composition add-on. The Factor F is then calculated as

Factor F=composition basis weight [g/m²]/average integrated peak area

If the composition does not contain a sufficient amount of linear hydrocarbon like material, or the substrate contains materials that do not allow for a quantification of composition between 4296 cm-1 and 4368 cm-1, a different wave number range in the infrared or near infrared range has to be identified that is suitable to quantify the composition by IR spectroscopy. Any wave number range with a linear correlation between integrated absorption coefficient above base line and composition basis weight can be used. If more than one possible wave number range can be identified, the range with the best signal to noise ratio is used. Whenever the composition is based on linear hydrocarbon like materials with CH2 groups the absorption band between 4296 cm-1 and 4368 cm-1 should be used.

B. Relative Concentration of Composition on Surface Test Method

Relative concentration of a composition on a surface of the fibrous structure and/or sanitary tissue product may be determined by using near IR spectroscopy, especially if the sample contains a hydrocarbon-containing composition. The near IR spectroscopy method may use a filter photometer or other near IR instrument, but it must be configured for back scatter detection. Appropriate wavelengths are used.

The fibrous structure and/or sanitary tissue product is placed under the near IR instrument and a reading is obtained. The sample is then turned over to obtain a reading from the other side of the sample.

In addition to near IR, mid-IR spectroscopy with suitable equipment and wavelengths may also be used to determine relative concentration of a composition on a surface of a fibrous structure and/or sanitary tissue product.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A fibrous structure comprising a user contacting surface wherein the user contacting surface comprises a first region comprising a surface treating composition and second region comprising a lotion composition.

2. The fibrous structure according to claim 1 wherein the first region comprises a continuous or substantially continuous network and the second region comprises a plurality of discrete areas dispersed throughout the continuous or substantially continuous network.

3. The fibrous structure according to claim 1 wherein the first region comprises a plurality of discrete areas.

4. The fibrous structure according to claim 1 wherein the second region comprises a plurality of discrete areas.

5. The fibrous structure according to claim 1 wherein the first region and the second region are in contact with each other.

6. The fibrous structure according to claim 1 wherein the first region and second region are separate and discrete from each other.

7. The fibrous structure according to claim 1 wherein the user contacting surface comprises a portion wherein the first region is in contact with the second region and another portion wherein the first region is separate and discrete from the second region.

8. The fibrous structure according to claim 1 wherein at least the first region is associated with a surface of the fibrous structure.

9. The fibrous structure according to claim 8 wherein the first region is associated with the entire or substantially the entire surface area of the surface of the fibrous structure.

10. The fibrous structure according to claim 1 wherein the second region is associated with a surface of the fibrous structure.

11. The fibrous structure according to claim 1 wherein the second region is present on a surface of the first region such that the first region is positioned between the second region and a surface of the fibrous structure.

12. The fibrous structure according to claim 11 wherein the second region is present on less than all of the surface area of the surface of the first region.

13. The fibrous structure according to claim 11 wherein the second region is present on the first region in the form of separate, discrete islands.

14. The fibrous structure according to claim 8 wherein the surface treating composition of the first region is present on the surface of the fibrous structure at a greater level by weight than within the fibrous structure.

15. The fibrous structure according to claim 1 wherein the surface treating composition comprises a surface treating agent.

16. The fibrous structure according to claim 15 wherein the surface treating agent is selected from the group consisting of: polymers, hydrocarbons, waxes, oils, silicones, quaternary ammonium compounds, fluorocarbons, substituted C₁₀-C₂₂ alkanes, substituted C₁₀-C₂₂ alkenes, polyols, sugar derivatives and mixtures thereof.

17. The fibrous structure according to claim 16 wherein the silicones are selected from the group consisting of: polydimethylsiloxanes, aminosilicones, cationic silicones, quaternary silicones, silicone betaines and mixtures thereof.

18. The fibrous structure according to claim 16 wherein the polysiloxanes are selected from compounds having monomeric siloxane units of the following structure: 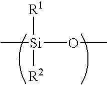
wherein, R¹ and R2, for each independent siloxane monomeric unit can each independently be hydrogen or any alkyl, aryl, alkenyl, alkaryl, arakyl, cycloalkyl, halogenated hydrocarbon, or other radical.

19. The fibrous structure according to claim 16 wherein the quaternary ammonium compounds are selected from compounds having the formula: 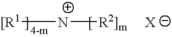
wherein:
m is 1 to 3; each R¹ is independently a C₁-C₆ alkyl group, hydroxyalkyl group, hydrocarbyl or substituted hydrocarbyl group, alkoxylated group, benzyl group, or mixtures thereof; each R² is independently a C₁₄-C₂₂ alkyl group, hydroxyalkyl group, hydrocarbyl or substituted hydrocarbyl group, alkoxylated group, benzyl group, or mixtures thereof; and X⁻ is any softener-compatible anion.

20. The fibrous structure according to claim 1 wherein the lotion composition comprises a compound selected from the group consisting of: hydrocarbons, fatty acid esters, alcohol ethoxylates and mixtures thereof.

21. The fibrous structure according to claim 1 wherein the lotion composition is a transferable lotion composition capable of being transferred to an opposing surface.

22. A fibrous structure comprising a surface treating composition and a lotion composition, wherein the surface treating composition is present on a surface of the fibrous structure at a greater level by weight than within the fibrous structure and the lotion composition is present within the fibrous structure at a greater level by weight than on the surface of the fibrous structure.

23. The fibrous structure according to claim 23 wherein the lotion composition is a transferable lotion composition capable of being transferred to an opposing surface.

24. The fibrous structure according to claim 22 wherein the lotion composition is present on less than the entire surface area of the surface of the fibrous structure.

25. The fibrous structure according to claim 22 wherein the surface treating composition is present on less than the entire surface of the surface of the fibrous structure.

26. The fibrous structure according to claim 22 wherein the surface of the fibrous structure comprises separate, discrete islands of the lotion composition.

27. The fibrous structure according to claim 22 wherein the lotion composition is present on a surface of the surface treating composition.

28. A single- or multi-ply sanitary tissue product comprising a fibrous structure according to claim 1.

29. A single- or multi-ply sanitary tissue product comprising a fibrous structure according to claim 22.

30. The sanitary tissue product according to claim 29 wherein the sanitary tissue product is a multi-ply sanitary tissue product and the lotion composition is present between two or more plies of the multi-ply sanitary tissue product.

31. A fibrous structure comprising:
a. a surface treating composition comprising a compound selected from the group consisting of: quaternary ammonium compounds, aminosilicones, polydimethylsiloxanes and mixtures thereof; and
b. a lotion composition comprising a compound selected from the group consisting of: oils, alcohol ethoxylates, fatty acid esters, hydrocarbons and mixtures thereof;
wherein the surface treating composition is present on a surface of the fibrous structure and the lotion composition is present on less than the entire surface of the surface treating composition.

32. A process for treating a fibrous structure comprising the step of applying a lotion composition to a surface treating composition associated with a surface of a fibrous structure.

33. A process for treating a fibrous structure comprising the steps of:
a. applying a surface treating composition to a surface of a fibrous structure; and
b. applying a lotion composition to the surface treating composition.

34. A process for treating a fibrous structure comprising the steps of:
a. applying a surface treating composition to a surface of a fibrous structure; and
b. applying a lotion composition to the surface of the fibrous structure.
